(19) United States
(12) Patent Application Publication
(10) Pub. No.: US 2017/0088642 A1
(43) Pub. Date: Mar. 30, 2017

(54) CATALYST COMPONENT FOR OLEFIN POLYMERIZATION AND CATALYST CONTAINING CATALYST COMPONENT AND USE THEREOF

(71) Applicant: Beijing Lihe Technology Ltd., Beijing (CN)

(72) Inventors: Zhiwu WANG, Beijing (CN); Shuhang LI, Beijing (CN); Huashu LI, Beijing (CN); Junwei ZHANG, Beijing (CN); Hui ZHANG, Beijing (CN); Yena CHEN, Beijing (CN); Jinsong DAI, Beijing (CN); Qingli Ma, Beijing (CN); Jingyu WANG, Beijing (CN); Shubin LI, Beijing (CN); Hao CHEN, Beijing (CN); Fengyao LEI, Beijing (CN); Wei BAI, Beijing (CN); Lige LI, Beijing (CN); Yong GAO, Beijing (CN)

(21) Appl. No.: 15/376,687

(22) Filed: Dec. 13, 2016

Related U.S. Application Data
(63) Continuation of application No. PCT/CN2014/080229, filed on Jun. 18, 2014.

(30) Foreign Application Priority Data
Jun. 13, 2014 (CN) 201410264392.2

(51) Int. Cl. C08F 10/06; C07C 69/753; C08F 10/02

(52) U.S. Cl. CPC C08F 10/06; C08F 10/02; C07C 69/753; C07C 2103/18

(57) ABSTRACT

A catalyst component for olefin polymerization, comprising Mg, Ti, a halogen and an electron donor, wherein the electron donor is at least one unsaturated ring-substituted diacid ester compound. Also provided is a catalyst containing the catalyst component and the use of the catalyst in an olefin polymerization, e.g., propylene polymerization.

### CROSS-REFERENCE TO RELATED APPLICATIONS

This application is a continuation of International Application PCT/CN2014/080229, filed Jun. 18, 2014, which claims priority to Chinese Application No. 201410264392.2, filed Jun. 13, 2014, the disclosure of each of which is incorporated by reference herein in its entirety.

### TECHNICAL FIELD

The present invention relates to a solid catalyst component for CH₂═CHR olefin polymerization, where R is hydrogen or hydrocarbon group having 1 to 12 carbon atoms, and more particularly, the present invention relates to a catalyst component containing at least one ring-substituted malonate compound, a catalyst containing the catalyst component and the use of the catalyst in reactions of olefin polymerization, particularly in the reactions of propylene polymerization.

### BACKGROUND ART

Electron donor compounds can maximally change the property of the active center of Ziegler-Natta catalysts for olefin polymerization, thereby changing the performance of the catalyst to the greatest extent. Therefore, in a sense, research on the Ziegler-Natta catalyst is to find a better electron donor. The research on the internal electron donor in China and abroad is mainly focused on traditional fatty acid esters and aromatic acid ester compounds; diethers (e.g. EP0361493, EP0728724) and succinic acid esters (e.g. WO9856834, WO0063261, WO03022894) compounds; and diol esters (e.g. CN1580033, CN1580034, CN1580035) compounds, etc. However, in practical applications, there are some problems with the aforementioned compounds serving as the electron donor of catalyst component for olefin polymerization, e.g. the polymers obtained by use of the catalyst system prepared by diether compounds have a narrow molecular weight distribution, while the polymer products obtained by use of the succinic acid ester catalyst system have a broad molecular weight distribution, the activity of diol esters catalyst system is often not as good as that of diether system. In order to obtain a more balanced overall performance of the catalyst, a variety of new compounds have been developed and used in the preparation of Ziegler-Natta catalysts.

However, Ziegler-Natta catalyst components prepared by using the aforementioned compounds are still unsatisfactory in activity/isotacticity when used for olefin polymerization, therefore further research and development are still required.

### SUMMARY OF THE INVENTION

The object of the present invention is to provide a catalyst component for CH₂═CHR olefin polymerization.

Another object of the present invention is to provide a method for preparing the catalyst component.

A further object of the present invention is to provide use of the catalyst component in preparation of a catalyst for CH₂═CHR olefin polymerization.

To attain the object of the present invention, provided is a catalyst component for olefin polymerization (CH₂═CHR olefin, where R is hydrogen or hydrocarbon group having 1 to 12 carbon atoms), which comprises Mg, Ti, a halogen and an electron donor. The electron donor is selected from at least one of unsaturated ring-substituted diacid ester compounds represented by the general formula (I) below:

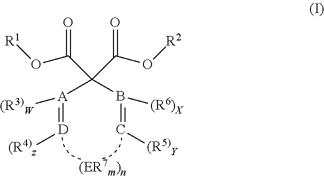

wherein, A, B, C, D, and E are each carbon atoms or heteroatoms selected from N, O and S; W, X, Y, Z, and m are each 0 or 1; with the proviso that

when n is equal to 0:

I) A, B, C and D are each carbon atoms, X, Y, Z and W are each 1; or

II) A is nitrogen atom, B, C and D are each carbon atoms, W is 0, X, Y and Z are each 1; or

III) A and D are each nitrogen atoms, B and C are each carbon atoms, W and Z are each 0, X and Y are each 1; or

IV) D is a nitrogen atom, A, B, and C are each carbon atoms, Z is 0, W, X and Y are each 1; or

when n is equal to 1:

i) A, B, C, D and E are each carbon atoms, m is 2, W, X, Y, and Z are 1; or

ii) E is a nitrogen atom, A, B, C and D are each carbon atoms, m is 1, W, X, Y and Z are each 1; or

iii) E is an oxygen atom, A, B, C and D are each carbon atoms, m is 0, W, X, Y and Z are each 1; or

iv) E is a sulfur atom, A, B, C, and D are each carbon atoms, m is 0, W, X, Y, and Z are each 1; or

v) D and E are nitrogen atoms, A, B and C are each carbon atoms, m is 1, W, X and Y are each 1, Z is 0;

R¹ and R² are the same or different C₁-C₂₀ hydrocarbon group, such as C₁-C₂₀ linear or branched alkyl, alkenyl, C₃-C₂₀ cycloalkyl, C₆-C₂₀ aryl, C₇-C₂₀ alkaryl and C₇-C₂₀ aralkyl group; R³-R⁷ are the same or different, and are each selected from hydrogen atom, halogen atom, oxygen atom, sulfur atom and C₁-C₂₀ hydrocarbon group, such as C₁-C₂₀ linear or branched alkyl, C₃-C₂₀ cycloalkyl, C₆-C₂₀ aryl, C₇-C₂₀ alkaryl and C₇-C₂₀ aralkyl group;

Said R¹-R⁷ each may optionally contain one or more R atoms as a substituent of carbon atom, a hydrogen atom, or both, where R is a heteroatom, a linear or branched C₁-C₂₀ alkyl, C₃-C₂₀ cycloalkyl, C₆-C₂₀-aryl, C₇-C₂₀ alkaryl or C₇-C₂₀ aralkyl group; wherein any two groups of R¹-R⁷ may be bonded to each other to generate one or more spiro ring or fused ring structures.

Examples of the compounds included in the general formula (I) are listed as follows:

diethyl 3,5-diphenyl 2H pyrrole-2,2-dicarboxylate; diethyl 3-(3-chlorophenyl)-5-methyl-pyrrole-2,2-dicarboxylate; diethyl 3-(3-bromophenyl)-5-methyl-pyrrole-2, 2-dicarboxylate; diethyl-3-3-(p-chlorophenyl)-5-phenyl-2H-pyrrole-2,2-dicarboxylate; dimethyl fluorene-9,9-dicarboxylate; diethyl fluorene-9,9-dicarboxylate; di-n-propyl fluorene-9,9-dicarboxylate; diisopropyl fluorene-9,9-dicarboxylate; di-n-butyl fluorene-9,9-dicarboxylate; di-isobutyl fluorene-9,9-dicarboxylate; di-n-pentyl fluorene-9,9-dicarboxylate; di-n-hexyl fluorene-9,9-dicarboxylate; di-n-heptyl fluorene-9, 9-dicarboxylate; di-n-octyl fluorene-9,9-dicarboxylate; 9-methyl carboxylate-9-ethyl carboxylate-fluorene; 9-methyl carboxylate-9-n-propyl carboxylate-fluorene; 9-methyl carboxylate-9-isopropyl carboxylate-fluorene; 9-methyl carboxylate-9-n-butyl carboxylate-fluorene; 9-methyl carboxylate-9-isobutyl carboxylate-fluorene; 9-ethyl carboxylate-9-n-propyl carboxylate-fluorene; 9-ethyl carboxylate-9-isopropyl carboxylate-fluorene; 9-ethyl carboxylate-9-n-butyl carboxylate-fluorene; 9-ethyl carboxylate-9-isobutyl carboxylate-fluorene-dimethyl 4H-benzo[g]thia<2,3-e> indazole-4,4-dicarboxylate; diethyl-5-phenyl-3 (p-toluene)-2H-pyrrole-2,2-carboxylate; diethyl-3 (p-methoxyphenyl)-5-phenyl-2H-pyrrole-2,2-dicarboxylate; diethyl 5-(p-nitro)-3-phenyl-2H-pyrrole-2,2-dicarboxylate; diethyl-2,3-diphenyl-1H-indene-1,1-dicarboxylate; diethyl-2-phenyl-1H-indene-1,1-dicarboxylate; diethyl-2-(4-chlorophenyl)-1H-indene-1,1-dicarboxylate; diethyl-2-(4-methoxyphenyl)-1H-indene-1,1-dicarboxylate; dimethyl 3-(4-methylphenyl)-2-phenyl-1H-indene-1,1-dicarboxylate; dimethyl-3-(4-nitrophenyl)-1H-indene-1,1-dicarboxylate; dimethylamino-4-pentamethoxycarbonyl-1,2,3,5,5-pentamethoxycarbonylcyclopentadiene; methyl 3-phenyl-indene-1,1-dicarboxylate; dimethyl-5-(p-chlorophenyl)3-phenyl-2H-pyrrole-2,2-dicarboxylate; dimethyl 3,4-di(p-chlorophenyl) 2H-pyrrole-dicarboxylate; dimethyl 3-(p-nitrophenyl)-5-phenyl-2H-pyrrole-2,2-dicarboxylate; dimethyl 3-(m-nitrophenyl)-5-phenyl-2H-pyrrole-2,2-dicarboxylate; dimethyl 5-(m-nitrophenyl)-5-phenyl-2H-pyrrole-2,2-dicarboxylate; dimethyl 5,6-dimethyl-5H,6H-cyclopentadiindole-11,11-dicarboxylate; 1-(2-nitrophenylthio)-2,3,4,5,5-methyl carboxylate-cyclopentadiene; 1-(2,4-dinitrophenyl)-2,3,4,5,5-methyl pentacarboxylate-cyclopentadiene; methyl-2-tert-butyl-3-methyl-1H-indene-1,1 dicarboxylate; dimethyl 3-methyl-2-trimethylsilyl-indene-1,1-dicarboxylate; dimethyl 3-methyl-2-phenyl-indene-1,1-dicarboxylate; diethyl-2,3-di-n-propyl-1H-indene-1, 1-dicarboxylate; dimethyl-3-hydroxymethyl-2-phenyl-1H-indene-1,1-dicarboxylate; dimethyl-2-tert-butyl-5, 6-dimethoxy-3-methyl-1H-indene-1,1-dicarboxylate; dimethyl-2-phenyl-3-(thia-2-yl)-1H-indene-1,1-dicarboxylate; dimethyl-3-(2-toluene)2-phenyl-1H-indene-1,1-dicarboxylate; dimethyl 3-(2-methoxycarbonylphenyl)-2-phenyl-1H-indene-1,1-dicarboxylate; dimethyl 3-(4-trifluoromethylphenyl) 2-phenyl-1H-indene-1,1-dicarboxylate; dimethyl 3-(4-acetylphenyl) 2-phenyl-1H-indene-1,1-dicarboxylate; dimethyl-2-(cyclohex-1-enyl)-3-(4-acetylphenyl)-1H-indene-1,1-dicarboxylate; dimethyl 2-[(ethoxycarbonyl)methyl]-1H-indene-1,1-dicarboxylic acid ester; 1,1-diethyl-1H-indene-1,1-dicarbonate; ethyl 7-chloro-5methyl-pyrazolo[4,3-d]pyrimidine-3,3-dicarbonate; ethyl 5-chloro-7-methyl-pyrazolo[4,3-d]pyrimidine-3,3-dicarbonate; ethyl 5-amino-7-methyl-pyrazolo[4,3-d]pyrimidine-3,3-dicarbonate; ethyl 7-methoxy-5-methyl-pyrazolo[4,3-d]pyrimidine-3,3-dicarbonate; 1-p-methylphenylamino-2,3,4,5,5-pentamethoxycarbonylcyclopentadiene; dimethyl-3H-phenanthro<9,10-c>pyrazole-3,3-dicarbonate; 3,3-di(methoxycarbonyl)-3H-indazole; 3,3-di(ethoxycarbonyl)3H-indazole; 1-trichloromethyl-2,3,4,5,5-pentamethoxycarbonylcyclopentadiene; 1-(2-methyl-4-nitrophenyl)-pentamethoxycarbonylcyclopentadiene; 1-(2-iodo-4-nitrophenyl)-pentamethoxycarbonylcyclopentadiene; 2-(2-iodo-4-nitrophenyl)-1,3,4,5,5-pentamethoxycarbonylcyclopentadiene; 1-(2,4-dinitrophenyl)-2,3,4,5,5-pentamethoxycarbonylcyclopentadiene; 4-benzyl-1,2,3,5,5-penta(methoxycarbonyl)cyclopentadiene; 3-benzyl-1,2,4,5,5-penta(methoxycarbonyl)cyclopentadiene; 2-(trifluoromethyl)-5-carbonyl-3,3-di(methoxycarbonyl)-3H-indole; 2-(trifluoromethyl)-5-carbonyl-7-methyl-3,3-di(methoxycarbonyl)-3H-indole; 3-(trifluoromethyl)-5-hydroxy-7-methoxy-3,3-di(methoxycarbonyl)-3H-indole; diethyl-3-phenyl-5(p-toluene)2H-pyrrole-2,2-dicarbonate; diethyl-2-(4-chlorophenyl)-5-morpholine-4H-imidazole-4,4-dicarbonate; 4,5,5-methyl tricarboxylate-1,2,3-trichlorocyclopentadiene; methyl-3-methyl-4-trimethylsilyl-cyclopenta-2,4-diene-1,1-dicarbonate; diethyl-2, 5-diphenyl-4H-imidazole-4,4-dicarbonate; diethyl-3-benzyl-2-phenyl-1H-indene-1,1-dicarbonate; diethyl-3-(4-(methoxycarbonyl)phenyl)2-phenyl-1H-indene-1,1-dicarbonate; diethyl-3-(4-acetylphenyl)2-phenyl-1H-indene-1,1-dicarbonate; diethyl-2-methoxymethyl-1H-indene-1,1-dicarbonate; dimethyl-2-tert-butyl-1H-indene-1,1-dicarbonate; diethyl-2-tert-butyl-1H-indene-1,1-dicarbonate; dimethyl 2-n-butyl-1H-indene-1,1-dicarbonate; diethyl 2-n-butyl-1H-indene-1,1-dicarbonate; diethyl 2-n-hexyl-1H-indene-1,1-dicarbonate; diethyl-2-(3-cyano-1-propyl)-1H-indene-1,1-dicarbonate; diethyl-2-diethoxymethyl-1H-indene-1,1-dicarbonate; diethyl-2-(4-methoxyphenyl)-1H-indene-1,1-dicarbonate; diethyl-2-(1-cyclohexene)-1H-indene-1,1-dicarbonate; diethyl-2-(cyclohexyl)-1H-indene-1,1-di carbonate; diethyl-3-(3-toluene)-2-phenyl-1H-indene-1,1-dicarbonate; diethyl-3-(3-nitrophenyl)-2-phenyl-1H-indene-1,1-dicarbonate; diethyl 13H-indeno[1,2-e]-phenanthrene-13,13-dicarbonate; diethyl-2-hexyl-3-(4-methoxyphenyl)1H-indene-1,1-dicarbonate; ethyl cyclopenta[c]thia-5,5-dicarbonate; 4-[4-[4-(methylsulfonic)phenyl]1,1-di(methoxy)cyclopenta-2,4-diene-3-yl]pyridine; fluorene-4,9,9-dicarboxylic acid-4-tert-butyl-9,9-dimethyl ester; methyl 4-(4-amino-pyridine-3-ylcarbamoyl)-fluorene-9,9-dicarbonate; dimethyl 4-(3-amino-pyridine-4-ylcarbamoyl)-fluorene-9,9-dicarbonate; diethyl-3-iodo-2-phenyl-1H-indene-1,1-dicarbonate; diethyl-3-iodo-2-n-pentyl-1H-indene-1,1-dicarbonate; diethyl-3-iodo-2-(3methoxyphenyl)-1H-indene-1,1-dicarbonate; diethyl-3-iodo-2-(naphthalen-2-yl)-1H-indene-1,1-dicarbonate; di-n-hexyl-fluorene-9,9-dicarbonate; di-n-heptyl-fluorene-9,9-dicarbonate; diethyl-2-phenyl-3H-indene-3,3-dicarbonate; diethyl-2-bromo-1H-indene-1,1-dicarbonate; 1-ethyl-1-methyl-cyclohexa-2,5-diene-1,1-dicarbonate; N,4,4-triethoxycarbonyl-1,4-dihydro-pyridine; 2,6-diphenyl-4,4-dimethoxycarbonyl-4H-pyrane; 2,6-diphenyl-4,4-dimethoxycarbonyl-1,4-dihydropyridine; 2,6-di(4-chlorophenyl)-4,4-dimethoxycarbonyl-4H-pyrane; 2,6-di(4-methoxyphenyl)-4,4-dimethoxycarbonyl-4H-pyrane; 2,6-di(4-chlorophenyl)-4,4-dimethoxycarbonyl-1,4-dihydropyridine; 2,6-di(4-methoxyphenyl)-4,4-dimethoxy carbonyl-1,4-dihydropyridine; 1-cyclopentyl-4,4-di(methoxycarbonyl)-1,4-dihydropyridine; 1-n-hexyl-4,4-di(methoxy carbonyl)-1,4-dihydropyridine; 1-methoxy-6,6-dimethylcarbonyloxymethyl-cyclohexa-1,4-diene; dimethyl 1,4-dihydronaphthalene-1,1-dicarbonate; 2,6-di(4-chlorophenyl)-4,4-dimethoxycarbonyl-4H-thiapyran; diethyl-3-bromo-1,4-dihydro-1-methylpyridazino[3,4-b]quinoxaline-4,4-dicarbonate; diethyl-5-bromo-3-phenyl-1,4-dihydropyridazine-4,4-dicarbonate; trihexyl-3-phenyl-1,4-dihydropyridazine-4,4,5-tricarboxylate; 1-phenethyl-di(methoxycarbonyl)1,4-dihydropyridine; diethyl-2-methyl-6-benzo(4H-pyran)4,4-dicarbonate; 1-(2-naphthylmethyl)-4,4-di(methoxycarbonyl)-1,4-dihydropyridine; dimethyl-3-acetyl-1-methylquinoline-4,4(1H)-dicarbonate.

Preferred compounds of the general formula (I) comprise the compounds of the following general formula (II):

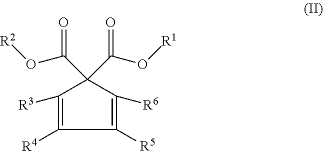

wherein R¹-R⁶ groups are defined as in the general formula (I), R³-R⁶ groups are the same or different.

Preferred compounds of the general formula (II) comprise the compounds of the following general formula (III):

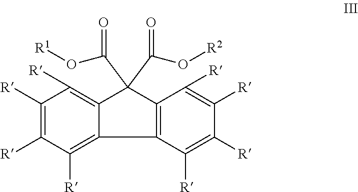

wherein R¹-R² groups are defined as in the general formula (I), R′ are the same or different, and each R′ is independently selected from hydrogen, halogen atom, linear or branched C₁-C₂₀ alkyl, C₃-C₂₀ cycloalkyl, C₆-C₂₀ aryl, C₇-C₂₀ alkaryl and C₇-C₂₀ aralkyl group.

In the five-membered ring compounds represented by the general formula (III), suitable specific compounds include the following:

dimethyl fluorene-9,9-dicarboxylate; diethyl fluorene-9, 9-dicarboxylate; di-n-propyl fluorene-9,9-dicarboxylate; diisopropyl fluorene-9,9-dicarboxylate; di-n-butyl fluorene-9,9-dicarboxylate; diisobutyl fluorene-9,9-dicarboxylate; di-n-pentyl fluorene-9,9-dicarboxylate; di-n-hexyl fluorene-9, 9-dicarboxylate; di-n-heptyl fluorene-9,9-dicarboxylate; di-n-octyl fluorene-9, 9-dicarboxylate; 9-methyl carboxylate-9-ethyl carboxylate-fluorene; 9-methyl carboxylate-9-n-propyl carboxylate-fluorene; 9-methyl carboxylate-9-isopropyl carboxylate-fluorene; 9-methyl carboxylate-9-n-butyl carboxylate-fluorene; 9-methyl carboxylate-9-isobutyl carboxylate-fluorene; 9-ethyl carboxylate-9-n-propyl carboxylate-fluorene; 9-ethyl carboxylate-9-isopropyl carboxylate-fluorene; 9-ethyl carboxylate-9-n-butyl carboxylate-fluorene; 9-ethyl carboxylate-9-isobutyl carboxylate-fluorene;

The unsaturated ring-substituted malonate of the present invention can be synthesized by a variety of reactions. One of them is a three-step reaction that comprises:

A) reacting a corresponding ring-substituted compound with carbon dioxide and alkyl lithium reagent, or with alkyl dimethyl ester and sodium hydride to obtain a cyclic hydrocarbon substituted carboxylic acid (see U.S. Pat. No. 4,564,700A1); B) reacting the product of Step A with a corresponding alcohol R¹OH to form a formate by esterification, or with a suitable ester precursor to directly form a cyclic hydrocarbon-substituted formate by addition (see Journal of the Chemical Society, 1949, P 2182, 2185); C) reacting the product of Step B with a precursor of a suitable haloformate by addition (see Analytical Chemistry, vol. 32, No. 4, April 1960).

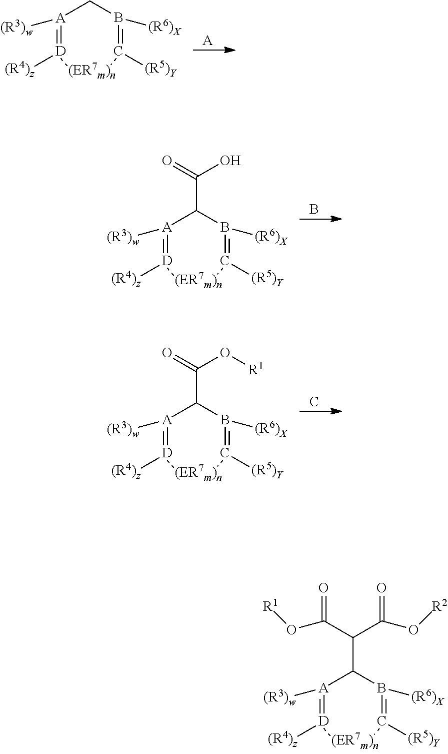

In addition, when the unsaturated ring-substituted diacid ester compound R¹ and R² are the same, the corresponding unsaturated ring-substituted compound can be directly reacted with a lithium reagent (such as n-butyllithium or lithium diisopropylamide) and a halogenated formate, each substituent is the same as defined in the general formula (I) (see Tetrahedron Letters 50 (2009) 6057-6059):

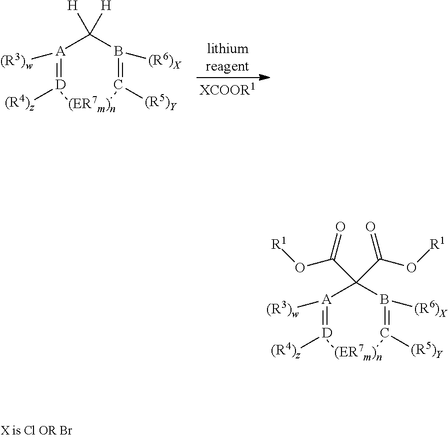

The catalyst component for olefin polymerization of the present invention comprises the reaction product of a titanium compound, a magnesium compound and ring-substituted malonate ester compound selected from the general formula (I)-(III), the precursor of said magnesium compound is selected from at least one of: Mg(OR)₂, XₙMg(OR)₂₋ₙ, MgCl₂.mROH, R₂₋ₙMgXₙ, MgR₂, MgCl₂/SiO₂, MgCl₂/Al₂O₃ or mixture of magnesium halide and titanium alkoxide, wherein m is a number from 0.1 to 6, 0<n<2, X is halogen, R is C₁-C₂₀ hydrocarbon group; said titanium compound is represented by general formula TiXₙ(OR)₄₋ₙ, wherein R is C₁-C₂₀ hydrocarbon group, X is halogen, n=1-4.

The magnesium compounds of the present invention are preferably magnesium hydrocarboxide compounds.

Other preferred magnesium compounds of the present invention are alcoholates of magnesium dihalide.

Yet other preferred magnesium compounds of the present invention are liquid magnesium compounds.

The titanium compounds of the invention include titanium tetrachloride, titanium tetrabromide, titanium tetraiodide and alkyl titanium halide, alkyl titanium halide such as methoxy titanium trichloride, ethoxy titanium trichloride, propoxy titanium trichloride, n-butoxy titanium trichloride, dimethoxy titanium dichloride, diethoxy titanium dichloride, dipropoxy titanium dichloride, di-n-butoxy dichloride titanium, trimethoxy titanium chloride, triethoxy titanium chloride, tripropoxy titanium chloride or tri-n-butoxy titanium chloride. These titanium halides can be used alone or in combination. A preferred titanium compound is titanium tetrachloride.

Preparation of the catalyst component of the present invention can be carried out according to several methods.

According to one of the methods, a solution of TiCl₄ or titanium alkoxide in an aromatic hydrocarbon (e.g., toluene, xylene, etc.), is reacted with magnesium dihydrocarboxide such as magnesium dialkoxide or magnesium diaryloxide or the like at −25-0° C., and halogenated at 80-130° C. Treatment with solution of TiCl₄ in an aromatic hydrocarbon can be repeated one or more times and the unsaturated ring-substituted diacid ester compounds of the general formula (I)-(III) can be added in such treatments. For example, the preparation can be carried out with reference to the preparation of titanium-containing solid catalyst component as disclosed in U.S. Pat. No. 5,077,357: successively adding magnesium ethoxide, titanium tetraethoxide, o-cresol, ethanol and chlorobenzene with stirring; quickly adding TiCl₄/chlorobenzene solution to the above liquid, heating the mixture until complete dissolution, continuing to heat the mixture up to a particular temperature; after using N₂ bubbling to remove the ethanol reactant, continuing stirring for a predetermined duration of time, and then washing with hot chlorobenzene, washing twice with isooctane, then drying by N₂ to obtain a carrier. Alternatively, the preparation can be done in accordance with another example: successively adding TiCl₄, titanium tetraethoxide, magnesium ethoxide and o-cresol in chlorobenzene with stirring; adding ethanol and keeping stirring at high temperature for 3 h until magnesium ethoxide is dissolved; hot filtering and washing with warm chlorobenzene and then with isooctane, finally drying by N₂.

According to another method, magnesium alkoxide or magnesium chloroalkoxide are reacted with an excess of TiCl₄ in a solution containing the unsaturated ring-substituted diacid ester compounds represented by the general formula (I)-(III) at a temperature of 80-135° C. According to a preferred method, the titanium compound represented by the general formula TiXₙ(OR)₄₋ₙ, wherein R is C₁-C₂₀ hydrocarbon group, X is halogen, n=1-4; preferably TiCl₄, is reacted with the adduct represented by the formula MgCl₂.mROH to prepare a solid catalyst component, wherein m is a number from 0.1 to 6, preferably from 2 to 3.5, and R is a hydrocarbon group having 1 to 20 carbon atoms. The adduct can be suitably prepared to be spherically shaped according to the following method: in the presence of an inert hydrocarbon which is immiscible with the adduct, alcohol and magnesium chloride are mixed, followed by quickly cooling the emulsion to solidify the adduct in a form of spherical particles. Examples of the spherical MgCl₂.mROH adduct prepared according to the method can be found in U.S. Pat. No. 4,399,054 and in U.S. Pat. No. 4,469,648. The obtained adduct can be directly reacted with the Ti compound or it can be first subjected to thermal controlled dealcoholation (80-130° C.) to obtain an adduct in which the mole number of alcohol is generally lower than 3, preferably between 0.1 and 2.5. The adduct (dealcoholated or itself) can then be suspended in cold TiCl₄ (generally −25-0° C.) to react with the titanium compound; the mixture was heated to 80-130° C. and kept at this temperature for 0.5-2 hours. Treatment with TiCl₄ can be repeated one or more times. During the treatment with TiCl₄, the unsaturated ring-substituted diacid ester compounds represented by the general formula (I)-(III) may be added and this treatment can be repeated one or more times.

Another method for preparing the catalyst component of the present invention includes the following steps: anhydrous magnesium chloride and the ring-substituted diacid ester compounds represented by the general formula (I)-(III) are grinded together under a condition that activation of the magnesium dichloride occurs. The product thus obtained can be treated with an excess of TiCl₄ at a temperature of 80-130° C. one or more times. After the above treatment the product is washed with a hydrocarbon solvent until no chlorine ions exist. According to a further method, the product obtained by co-grinding anhydrous magnesium dichloride, titanium compound and the unsaturated ring-substituted diacid ester compounds represented by the general formula (I)-(III) is treated with a halogenated hydrocarbon such as 1,2-dichloro ethane, chlorobenzene, methylene chloride or the like. This treatment is carried out at a temperature from 40° C. to boiling point of the halogenated hydrocarbon for 1-4 hours. Then the product can be obtained generally by washing with an inert hydrocarbon solvent such as hexane.

According to another method, magnesium dichloride is preactivated according to a known method, and then treated with an excess of TiCl₄ at a temperature of about 80-135° C., wherein the solution contains unsaturated ring-substituted diacid ester compounds represented by the general formula (I)-(III). The solid is treated with TiCl₄ repeatedly and washed with hexane to remove any unreacted TiCl₄.

A further method comprises the preparation carried out with reference to the preparation of titanium-containing solid catalyst component as disclosed in CN1208045: in the presence of one compound selected from alcohols, phenols, ketones, aldehydes, ethers, amines, pyridine and esters, a liquid magnesium compound is contacted with the liquid titanium compound to precipitate a solid at a low temperature, the temperature of contact is usually at −70 −200° C., preferably-30-130° C., during contact, a unsaturated ring-substituted diacid ester compounds represented by the general formula (I)-(III) is added for treatment.

Another method of the catalyst component of the present invention comprises: a magnesium compound is dissolved in a solvent system consisting of an organic epoxy compound, organophosphorus compound and an inert diluent composition to form a homogeneous solution, which is mixed with the titanium compound to precipitated a solid in the presence of co-precipitation agent; the solid is treated with a unsaturated ring-substituted diacid ester compound represented by the general formula (I)-(III) to allow the unsaturated ring-substituted diacid ester compound to load on the solid, if necessary, the thus-obtained product is then treated with titanium tetrahalide and an inert diluent, wherein the co-precipitating agent is one of organic acid anhydride, organic acid, ether and ketone. Among the components, based on per mol of magnesium halide, organic epoxy compound is 0.2 to 10 mol, organophosphorus compound is 0.1 to 3 mol, co-precipitation agent is 0.03 to 1.0 mol, halides and derivatives of transition metal Ti are 0.5 to 150 mol.

The catalyst component of the present invention can also be prepared by using an inorganic oxide, such as such as SiO₂, alumina or a porous resin, which is pre-loaded with a magnesium compound as a carrier and activated by known methods, and then treating the loaded carrier with an excess of TiCl₄ at a temperature of about 80-135° C., wherein an unsaturated ring-substituted diacid ester compounds represented by the general formula (I)-(III) is added during treatment.

The above reactions result in the formation of magnesium halide in an active form. In addition to these reactions, there are other known methods in the literature which start with a compound different from the magnesium halide to form magnesium halide in an active form.

In any preparation method, the unsaturated ring-substituted diacid ester compounds represented by the general formula (I)-(III) can be directly added or obtained through an optional manner, for example, by use of appropriate precursors to prepare in situ, the appropriate precursors can complete the conversion in the presence of suitable electron donor compounds, for example, by esterification, transesterification and other known chemical reactions. Typically, MgCl₂ and unsaturated ring-substituted diacid ester compounds represented by the general formula (I)-(III) are used in a molar ratio of 0.01-5, preferably 0.05-2.0.

The catalyst component of the present invention is converted into a catalyst for olefin polymerization by reaction with an organic aluminum compound according to known methods. In particular, one object of the present invention is to provide a catalyst for olefin CH₂═CHR polymerization, wherein R is hydrogen or hydrocarbon group having 1-12 carbon atoms, the catalyst comprising the reaction product of the following materials:

(a) a catalyst component of the present invention comprising Mg, Ti and a halogen and an unsaturated ring-substituted diacid ester compound represented by the general formula (I)-(III);

(b) at least one organic aluminum compound of the general formula AlRₙX₍₃₋ₙ₎, wherein R is hydrogen, hydrocarbon group having 1-20 carbon atoms; X is halogen, n is an integer of 0≦n≦3; and, optionally,

(c) at least one external electron donor compound.

Preferably, the organoaluminum compound (b) is selected from the group consisting of trialkylaluminum compound such as trimethylaluminum, triethylaluminum, triisobutylaluminum, tri-n-butyl aluminum, tri-n-hexyl aluminum, trioctyl aluminum. It is also possible to use trialkylaluminum and alkylaluminum halide, or a mixture of alkylaluminum sesquichloride such as AlEt₂Cl and Al₂Et₃Cl₃, alkylalumoxanes can also be used.

For applications where good isotacticity is required, an external electron donor compound can be used. The external electron donor is selected from siloxane compounds represented by general formula RₙSi(OR₁)₄₋ₙ, wherein R and R₁ are C₁-C₁₈ hydrocarbon group, which may optionally be substituted by heteroatoms; n is an integer of 0≦n≦3.

Said specific silane compounds may be: trimethylmethoxysilane, trimethylethoxysilane, tri-n-propylmethoxysilane, tri-n-propylethoxysilane, tri-n-butylmethoxysilane, triisobutylethoxysilane, trihexylmethylsilane, trihexylethoxysilane, dimethyldimethoxysilane, dimethyldiethoxysilane, di-n-propyldimethoxysilane, diisopropyldimethoxysilane, di-n-propyldiethoxysilane, diisopropyldiethoxysilane, di-n-butyldiethoxysilane, diisobutyldiethoxysilane, di-tert-butyldimethoxysilane, di-tert-butyldimethoxysilane, di-n-butyldimethoxysilane, diisobutyldimethoxysilane, di-tert-butyldiethoxysilane, di-n-butyldiethoxysilane, n-butylmethyldimethoxysilane, di(2-ethylhexyl)dimethoxysilane, di(2-ethylhexyl)diethoxysilane, dicyclohexyldimethoxysane, dicyclohexyldiethoxysilane, dicyclopentyldimethoxysilane, dicyclopentyldiethoxysilane, cyclohexylmethyldimethoxysilane, cyclohexylmethyldiethoxysilane, cyclohexylethyldimethoxysilane, cyclohexylisopropyldimethoxysilane, cyclohexylethyldiethoxysilane, cyclopentylmethyldimethoxysilane, cyclopentylethyldiethoxysilane, cyclopentylisopropyldiethoxysilane, cyclopentylisobutyldimethoxysilane, cyclohexyln-propyldimethoxysilane, cyclohexyln-propyldiethoxysilane, cyclohexyln-butyldiethoxysilane, pentylmethyldimethoxysilane, pentylmethyldiethoxysilane, pentylethyldimethoxysilane, pentylethyldiethoxysilane, cyclohexyldimethylmethoxysilane, cyclohexyldiethylmethoxysilane, cyclohexyldiethylmethoxysilane, cyclohexyldiethylethoxysilane, 2-ethylhexyltrimethoxysilane, cyclohexyldimethoxysilane, cyclohexyldiethoxysilane, 2-ethylhexyltriethoxysilane, ethyltrimethoxysilane, ethyltriethoxysilane, n-propyltrimethoxysilane, n-propyltriethoxysilane, isopropyltrimethoxysilane, isopropyltriethoxysilane, n-butyltrimethoxysilane, isobutyltrimethoxysilane, tert-butyltrimethoxysilane, n-butyltri ethoxysilane, cyclohexyltrimethoxysilane, cyclohexyltriethoxysilane, cyclopentyltrimethoxysilane, cyclopentyltriethoxysilane, vinyltrimethoxysilane, vinyltriethoxysilane, 2-ethylhexyltrimethoxysilane, 2-ethylhexyltriethoxysilane, pentyltrimethoxysilane, pentyltriethoxysilane, tetramethoxysilane, tetraethoxysilane, cyclohexyl cyclopentyl dimethoxysilane, cyclohexylcyclopentyldiethoxysilane, cyclohexylcyclopentyldipropoxysilane, 3-methylcyclohexylcyclopentyldimethoxysilane, 4-methylcyclohexylcyclopentyldimethoxysitane, 3,5-dimethylcyclohexylcyclopentyldimethoxysilane, 3-methylcyclohexylcyclohexyldimethoxysilane, di (3-methylcyclohexyl)dimethoxyslime, 4-methylcyclohexylcyclohexyldimethoxysilane, di(4-methylcyclohexyl)dimethoxysilane, 3,5-dimethylcyclohexylcyclohexyldimethoxysilane, di(3,5-dimethylcyclohexyl)dimethoxysilane, tetrapropoxysilane, tetrabutoxysilan. The preferable compound among these organosilicon compounds are: di-n-propyldimethoxysilane, diisopropyldimethoxysilane, di-n-butyldimethoxysilane, diisobutyldimethoxysilane, di-tert-butyldimethoxysilane, di-n-butyldiethoxysilane, tert-butyltrimethoxysilane, dicyclohexyldimethoxysilane, dicyclohexyldiethoxysilane, cyclohexylmethyldimethoxysilane, cyclohexylethyldiethoxysilane, cyclohexylethyldimethoxysilane, cyclohexylethyldiethoxysilane, cyclopentylmethyldimethoxysilane, cyclopentylmethyldiethoxysilane, cyclopentylethyldimethoxysilane, cyclohexylcyclopentyldimethoxysilane, cyclohexylcyclopentyldiethoxysilane, 3-methylcyclohexylcyclopentyldimethoxysilane, 4-methylcyohexylcyclopentyldimethoxysane and 3,5-dimethylcyclopentyldimethoxysilane, etc. These compound C can be used alone or in combination.

Preferred examples of silicon compounds are cyclohexylmethyl dimethoxysilane; diisopropyl dimethoxysilane; di-n-butyl dimethoxysilane; diisobutyl dimethoxysilane; diphenyl dimethoxysilane; phenyltriethoxysilane; methyl tert-butyl dimethoxysilane; dicyclopentyl dimethoxysilane; 2-ethylpiperidin-2-t-butyl-dimethoxysilane and (1,1,1-trifluoro-2-propyl)-2-ethylpiperidine dimethoxysilane and (1,1,1-trifluoro-2-propyl)-methyldimethoxysilane, cyclohexyl trimethoxysilane; tert-butyl trimethoxysilane and tert-hexyl trimethoxysilane.

The catalysts of the present invention can be used for olefin CH₂═CHR(co)polymerization, wherein the olefin is ethylene, propylene, 1-butene, 4-methyl-1-pentene, 1-hexene and 1-octene.

In order to use the catalysts of the present invention for olefin polymerization, the catalyst prepared by component a, b, c can be used for both homo-polymerization and co-polymerization. Typically the molar ratio of component b to component a is 1-1000 mol per mol of titanium atom contained in the component a, preferably 50-800 mol per mol of titanium atom contained in the component a; and the molar ratio of component c to component a is 0.002-10, preferably 0.01-2, more preferably 0.01-0.5.

The addition order of the components is arbitrary. Preferably, component b is firstly added to the polymerization system, and then component C, and component a is added last.

The polymerization process of the present invention can be carried out in the presence or absence of a solvent. Olefin monomers may be gaseous or liquid phase.

Hydrogen can be further added as a molecular weight modifier. Of course, the polymerization can also be carried out in the absence of molecular weight modifier. The polymerization temperature is not higher than 200° C., preferably is 20-100° C., and more preferably 40-80° C. The polymerization pressure is no more than 10 MPa, and is preferably 1-5 MPa. Both continuous polymerization and batch polymerization process can be applied. The polymerization reaction can be divided into one, two or more stages.

The olefins to be homopolymerized or copolymerized using the catalyst of the present invention include, linear olefins: ethylene, propylene, 1-butene, 1-pentene, 1-hexene, 1-heptene, 1-nonene, 1-decene; branched olefins such as: 3-methyl-1-butene and 4-methyl-1-pentene; dienes such as: butadiene, vinyl cyclopentene and vinyl cyclohexene. The catalyst of the present invention is preferably used for the polymerization of polyethylene and polypropylene. These olefins may be used alone or in combination.

In terms of the olefin polymerization conducted by using the catalyst component a, b, c of the present invention (hereinafter referred to as the main polymerization), prepolymerization is recommended to increase the activity of the catalysts as well as the isotacticity, particle properties of the product polymers. The prepolymerization can also be used for styrene homopolymerization.

In the prepolymerization process, the addition order of each component and monomer is arbitrary. Preferably the component b is firstly added to the system containing an inert gas or olefins to be polymerized, and then one or more olefins to be polymerized are added after addition of component a. In the process of olefin prepolymerization using organosilane, it is recommended that component b is added to the polymerization system of an inert gas or olefins to be polymerized, followed by the addition of component C, which is then followed by the addition of component a, and the olefins are added last.

The present invention utilizes bifunctional compounds having a specific structure, i.e., ring-substituted diacid ester compounds as shown in the general formula (I), since the oxygen of the ester bond has a strong coordination effect and is relatively stable during the preparation of the catalyst, therefore playing an positive and effective role in the catalyst activity and isotacticity of the polymer product.

The specific ring-substituted structure of the compounds of the present invention has a steric effect and can dictate the stereo-configuration of ester bifunctional groups, thus having a positive effect in the formation of the catalyst active sites and improvement of the stereospecificity of the catalyst.

The present inventors have found in experiments that, when these compounds are used as an electron donor to prepare a Ziegler-Natta catalyst component, the catalyst component has an excellent activity and a polyolefin product having a high isotacticity can be obtained. The compounds of the invention are applied to several most representative Ziegler-Natta catalyst preparation systems including magnesium ethylate system, magnesium chloride alcoholate system and magnesium chloride dissolution and precipitation system and other major systems, respectively, the resulting catalysts have a high compound content, indicating that the compounds have good coordination performance and stability; the resulting catalysts are generally higher in activity than the catalysts prepared using traditional aromatic diester electron donor under the same conditions, and has a high stereospecificity.

### SPECIFIC MODES FOR CARRYING OUT THE EMBODIMENTS

The following examples further illustrating the present invention are intended to make the advantages and effects of the invention better understood, but these examples are only for illustrating the present invention and not for limiting the present invention.

### Preparation of Unsaturated Ring-Substituted Diacid Ester Compounds

### Example 1 Synthesis of fluorene-9methyl carboxylate-9-ethyl carboxylate

Step A: to a 1000 mL three-necked flask were successively added 18 g sodium hydride, 50 g fluorene, 150 mL toluene under nitrogen, with mechanical stirring, the temperature was raised to 125° C. to reflux for 4 h; after cooling to 90° C., 146.1 g diethyl carbonate was slowly dropped to the flask over 1.5 h, then the reaction was continued for 3 h; after cooling to 20° C., a mixture of 60 g concentrated hydrochloric acid and 75 g water was slowly added dropwise, and the temperature was controlled to be no greater than 40° C.; the organic phase was separated by filtering and washed with water to neutral, followed by rotary evaporation to yield a red-brown liquid; the resulting liquid obtained by rotary evaporation, 157.4 g acetic acid and 63 g 10% hydrochloric acid were refluxed overnight; the mixture was cooled to 20° C., followed by liquid separation; 30% NaOH solution was added to the organic phase after rotary evaporation, which was adjusted to pH 8-9 and extracted with ethyl acetate, the aqueous phase was retained. Concentrated hydrochloric acid was added to the aqueous phase to adjust the pH to 5-6, which was extracted with ethyl acetate, the organic phase was retained for rotary evaporation; the products were dissolved in ethyl acetate and frozen for recrystallization; the crude products after filtration were washed with hexane to colorless crystals of about 10 g, melting point: 228˜230° C.

Step B: to a 250 mL three-necked flask were added 2 g (9.5 mmol) 9-fluorene carboxylic acid, methanol (30 mL), concentrated sulfuric acid (0.2 mL); the mixture was heated to reflux for 2 h; cooled to room temperature; poured into saturated sodium bicarbonate solution, and extracted twice with ethyl acetate (30 mL*2), the combined organic phase was washed with brine (30 mL*1), evaporated under reduced pressure to give a yellow solid, followed by drying with oil pump to give 1.8 g crude products with mp 62-65° C.

Step C: to a 250 mL three-necked round bottom flask were added methanol (20 mL), metallic sodium (0.12 g, 5 mmol) and placed under ice-bath, after metallic sodium was completely dissolved until no bubble emerges, methyl fluorene-9-carboxylate (0.56 g, 2.5 mmol) was added and completely dissolved, the mixture appeared yellow and was stirred for 5 min, ethyl chlorocarbonate (0.8 g, 7.5 mmol) was added therein, stirred for 30 min, poured into an aqueous solution, extracted with dichloromethane (20 mL*2) and extracted twice with ethyl acetate (50 mL*2). The combined organic phases were washed with saturated brine (50 mL*1), followed by rotary evaporation to remove liquid, the resulting crude product was washed with hexane and recrystallized from petroleum ether to give the product, 106-109° C.

fluorene-9-methyl carboxylate-9-ethyl carboxylate, 1H-NMR (CDCl₃) δ (ppm): 0.982-1.014 (t, 3H, CH₃), 3.758 (s, 3H, OCH₃), 4.130-4.156 (m, 2H, OCH₂), 7.356-7.388 (t, 2H, ArH), 7.439-7.470 (t, 2H, ArH), 7.714-7.728 (d, 2H, ArH), 7.790-7.7804 (d, 2H, ArH).

### Example 2 Synthesis of diethyl fluorene-9,9-dicarboxylate

A solution of n-butyl lithium/hexane (1.6 M, 15 mmol) was added dropwise to a 20 mL tetrahydrofuran solution containing 16 mmol of diisopropylamine at −78° C. The solution was stirred at −78° C. for 45 minutes, stirred at 0° C. for 20 minutes and then cooled to −78° C. A solution of 20 mL of tetrahydrofuran containing 7.0 mmol of fluorene was added dropwise to the stirred solution over a period of 30 minutes at −78° C. and 33 mmol of ethyl chloroformate was added to the mixture. The reaction system was allowed to warm to room temperature and stirred at room temperature for 3 hours. The reaction mixture was poured into 100 mL of water and extracted with ether (three times, with 50 mL of ether each time). The organic phase was dried over magnesium sulfate and concentrated. The crude product was recrystallized from petroleum ether to give the product, 100-101° C.

1H-NMR (CDCl₃) δ (ppm) of diethyl of fluorene-9,9-dicarboxylate: 0.932-0.962 (t, 6H, CH₃), 4.132-4.158 (m, 4H, O CH₂), 7.392-7.424 (t, 2H, ArH), 7.448-7.480 (t, 2H, ArH), 7.734-7.748 (d, 2H, ArH), 7.890-7.906 (d, 2H, ArH).

### Example 3 Synthesis of dimethyl fluorene-9,9-dicarboxylate

The preparation steps were the same as those in Example 2, except that the ethyl chloroformate was replaced by methyl chloroformate.

1H-NMR (CDCl₃) δ (ppm) of dimethyl of fluorene-9,9-dicarboxylate: 3.759 (s, 6H, CH₃), 7.359-7.392 (t, 2H, ArH), 7.443-7.475 (t, 2H, ArH), 7.720-7.735 (d, 2H, ArH), 7.799-7.7814 (d, 2H, ArH).

### Example 4 Synthesis of di-n-propyl fluorene-9,9-dicarboxylate

The preparation steps were the same as those in Example 2, except that the ethyl chloroformate was replaced by n-propyl chloroformate.

1H-NMR (CDCl₃) δ (ppm) of di-n-propyl fluorene-9,9-dicarboxylate: 0.936-0.966 (t, 6H, CH₃), 1.664-1.735 (m, 4H, CH₂), 4.171-4.197 (t, 4H, OCH₂), 7.389-7.421 (t, 2H, ArH), 7.449-7.481 (t, 2H, ArH), 7.737-7.752 (d, 2H, ArH), 7.887-7.902 (d, 2H, ArH).

### Example 5 Synthesis of diisopropyl fluorene-9,9-dicarboxylate

The preparation steps were the same as those in Example 2, except that the ethyl chloroformate was replaced by isopropyl chloroformate.

1H-NMR (CDCl₃) δ (ppm) of diisopropyl fluorene-9,9-dicarboxylate: 1.282-1.295 (t, 12H, CH₃), 5.012-5.062 (m, 4H, OCH), 7.215-7.295 (t, 2H, ArH), 7.307-7.354 (t, 2H, ArH), 7.356-7.371 (d, 2H, ArH), 7.654-7.686 (d, 2H, ArH).

### Example 6 Synthesis of di-n-butyl fluorene-9,9-dicarboxylate

The preparation steps were the same as those in Example 2, except that the ethyl chloroformate was replaced by n-butyl chloroformate.

1H-NMR (CDCl₃) δ (ppm) of di-n-butyl fluorene-9,9-dicarboxylate: 0.937-0.967 (t, 6H, CH₃), 1.363-1.438 (m, 4H, CH₂), 1.642-1.699 (m, 4H, CH₂), 4.220-4.246 (t, 4H, OCH₂), 7.394-7.426 (t, 2H, ArH), 7.447-7.479 (t, 2H, ArH), 7.734-7.749 (d, 2H, ArH), 7.889-7.904 (d, 2H, ArH).

### Example 7 Synthesis of diisobutyl fluorene-9,9-dicarboxylate

The preparation steps were the same as those in Example 2, except that the ethyl chloroformate was replaced by isobutyl chloroformate.

1H-NMR (CDCl₃) δ (ppm) of diisobutyl fluorene-9,9-dicarboxylate: 0.919-0.932 (d, 12H, CH₃), 1.936-2.016 (m, 2H, CH), 3.982-3.995 (d, 4H, OCH₂), 7.372-7.405 (t, 2H, ArH), 7.440-7.473 (t, 2H, ArH), 7.728-7.743 (d, 2H, ArH), 7.868-7.883 (d, 2H, ArH).

### Example 8 Synthesis of dibenzyl fluorene-9,9-dicarboxylate

The preparation steps were the same as those in Example 2, except that the ethyl chloroformate was replaced by benzyl chloroformate.

1H-NMR (CDCl₃) δ (ppm) of dibenzyl fluorene-9,9-dicarboxylate: 5.186-5.212 (s, 4H, CH₂), 7.372-7.405 (t, 2H, ArH), 7.384-7.426 (t, 6H, ArH), 7.440-7.473 (t, 2H, ArH), 7.478-7.602 (d, 4H, ArH), 7.728-7.743 (d, 2H, ArH), 7.868-7.883 (d, 2H, ArH).

TABLE 1
Summary of examples of ring-substituted fluorene diacid ester

| Example | Substituens | | |
|---|---|---|---|
| No. | R¹ | R² | Product |
| 1 | Me | Et | fluorene-9-methyl carboxylate-9-ethyl |
| | | | carboxylate |
| 2 | Me | Me | diethyl fluorene-9,9-dicarboxylate |
| 3 | Et | Et | dimethyl fluorene-9,9-dicarboxylate |
| 4 | ⁿPr | ⁿPr | di-n-propyl fluorene-9,9-dicarboxylate |
| 5 | ⁱPr | ⁱPr | diisopropylfluorene-9,9-dicarboxylate |
| 6 | ⁿBu | ⁿBu | di-n-butyl fluorene-9,9-dicarboxylate |
| 7 | ⁱBu | ⁱBu | diisobutylfluorene-9,9-dicarboxylate |
| 8 | Benzyl | Benzyl | dibenzyl fluorene-9,9-dicarboxylate |

Preparation of Solid Catalyst Component

Preparation of the catalysts in these Examples was carried out under high purity nitrogen. Specific examples were provided as follows.

### Example 9

To a 500 ml fully nitrogen-purged five-necked flask equipped with a stirrer were added 10 g diethoxy magnesium and 80 mL toluene to prepare a suspension, and then 20 mL titanium tetrachloride was added dropwise at −15° C., after addition was completed the system was slowly warmed to 10° C. after 60 mL titanium tetrachloride was added dropwise, then slowly warmed to 80° C. and then, 3.5 g fluorene-9-methyl carboxylate-9-ethyl carboxylate was added, and then the system was warmed up to 120° C. and maintained at the temperature for 2 hours, then the liquid was cleaned by filter pressing and filtered, the resulting solid was washed 3 times with 120 mL titanium tetrachloride at 125° C. The resulting solid was washed two times at 60° C. and two times at room temperature with 150 mL hexane, after filtering to remove the liquid and drying the solid, 10.34 g solid powder was obtained, i.e. solid catalyst component, the titanium content of which was determined to be 3.96 (wt) %, and the fluorene diacid ester content was determined to be 10.29 (0%.

### Example 10

To a 500 ml of fully nitrogen-purged five-necked flask equipped with a stirrer were added 10 g of MgCl₂.2.5C₂H₅OH microspheres and 150 mL titanium tetrachloride to prepare a suspension, and then the system was kept at −15° C. for 1 hour and warmed to 80° C., 4 g fluorene-9-methyl carboxylate-9-ethyl carboxylate was added, and then the system was warmed up to 110° C. and maintained at the temperature for 1 hour, then the liquid was cleaned by filter pressing and filtered, the resulting solid was washed 3 times with 120 mL titanium tetrachloride at 125° C. The resulting solid was washed four times with 150 mL hexane at 60° C., after filtering to remove the liquid and drying the solid, 4.73 g solid powder was obtained, i.e. catalyst component, the titanium content of which was determined to be 3.15 (wt) %, and the fluorene diacid ester content was determined to be 13.46 (wt) %.

### Example 11

7.1 g anhydrous magnesium chloride, 38 mL decane and 35 mL 2-ethylhexanol were reacted at 130° C. for 2 hours to form a homogeneous solution. 1.7 g phthalic anhydride was added to the solution, and stirred for 1 hour at 130° C. to completely dissolve phthalic anhydride in the homogeneous solution. The resulting homogeneous solution was cooled to room temperature and was dropwise added to 200 mL titanium tetrachloride kept at −20° C. over 1 hour; After addition was completed, the mixed solution was heated to 110° C. over 4 hours. When the temperature reached 110° C., 5 g fluorene-9-methyl carboxylate-9-ethyl carboxylate was added, the mixture was stirred at that temperature for 2 hours. After the reaction, the solid portion was collected by hot filtration. The solid portion was suspended in 275 mL titanium tetrachloride and reacted at 110° C. for 2 hours. After the reaction, the solid was collected by hot filtration, sufficiently washed with decane and hexane at 110° C., followed by suction filtration to give a catalyst component, the titanium content of which was determined to be 2.49 (wt) %, and the content of fluorene diacid ester was determined to be 10.24 (wt) %.

### Example 12

To a 500 ml fully nitrogen-purged five-necked flask equipped with a stirrer were added 10 g anhydrous magnesium chloride, 150 mL toluene, 17 mL epichlorohydrin and 16 mL tributyl phosphate at the room temperature, warmed to 50° C. with stirring and maintained for 2 hours until the solid was completely dissolved, and then 2.40 g phthalic anhydride was added, the reaction was maintained for 1 hour. The solution was cooled to −25° C., 110 mL titanium tetrachloride was dropwise added over a period of 1 hour, the temperature was slowly raised to 80° C., in the heating process, the solid was precipitated stepwise. 5 g fluorene-9-methyl carboxylate-9-ethyl carboxylate was added and the reaction was maintained at 80° C. for 1 hour. The resulting sold after filtration was washed twice with 200 mL toluene, and then 120 mL toluene and 80 mL titanium tetrachloride were added, the temperature was raised to 110° C. and maintained for 2 hours, then the liquid was cleaned by filter pressing, and the treatment was repeated one time. The resulting solid after filtration was washed one time with 100 mL dichloroethane, four times with hexane, and dried to give 9.7 g solid powder, i.e. the solid catalyst component, the titanium content of which was determined to be 4.81 (wt) %, and the fluorene diacid ester content was determined to be 14.81 (wt) %.

### Examples 13-19

Preparation steps of catalyst component were the same as described in Example 9, except that the fluorene-9-methyl carboxylate-9-ethyl carboxylate was replaced by diethyl fluorene-9,9-dicarboxylate, dimethyl fluorene-9,9-dicarboxylate, di-n-propyl fluorene-9,9-dicarboxylate, diisopropyl fluorene-9,9-dicarboxylate, diethyl fluorene-9,9-dicarboxylate, di-n-butyl fluorene-9, 9-dicarboxylate, diisobutyl fluorene-9,9-dicarboxylate or dibenzyl fluorene-9,9-dicarboxylate, respectively.

### Examples 20-21

Preparation steps of catalyst component were the same as described in Example 10, except that the fluorene-9-methyl carboxylate-9-ethyl carboxylate was replaced by diethyl fluorene-9,9-dicarboxylate or dimethyl fluorene-9,9-dicarboxylate, respectively.

### Examples 22-23

Preparation steps of catalyst component were the same as described in Example 11, except that fluorene-9-methyl carboxylate-9-ethyl carboxylate was replaced by diethyl fluorene-9,9-dicarboxylate or dimethyl fluorene-9,9-dicarboxylate, respectively.

Polymerization

Polymerization evaluation was made by using a catalyst as the catalyst component for olefin polymerization:

To a 5 L fully nitrogen-purged stainless steel reactor were added 5 mL solution of triethylaluminum in hexane at a concentration of 0.5 mol/L and 1 mL solution of methyl cyclohexyl dimethoxy silane (CMMS) in hexane at a concentration of 0.1 mol/L and 10 mg prepared catalyst, 10 mL hexane was added to rinse the feed lines, and then 2 L hydrogen (standard state) and 2.5 L purified propylene were added, the reaction was controlled at 20° C. to prepolymerize for 5 minutes, the temperature was raised to 70° C., at this temperature the polymerization reaction was carried out for 1 hour. After the reaction, the reactor was cooled and the stirring was stopped, the reaction product was discharged and dried to obtain a polymer. (Bulk density of the polymer is measured by JB/T 2412-2008 method, and the isotacticity was measured by JB/T 3682-2000 method.)

TABLE 2
Catalyst performance

| | | | | Activity | isotac- | Bulk |
|---|---|---|---|---|---|---|
| Example | internal electron donor | | titanium | Kg/ | ticity | density |
| No. | compound | Wt % | Wt % | gCat · h⁻¹ | % | g/cm³ |
| 9 | fluorene-9-methyl carboxylate-9-ethyl | 10.29 | 3.96 | 54 | 97.2 | 0.368 |
| | carboxylate | | | | | |
| 10 | fluorene-9-methyl carboxylate-9-ethyl | 13.46 | 3.15 | 62 | 97.5 | 0.366 |
| | carboxylate | | | | | |
| 11 | fluorene-9-methyl carboxylate-9-ethyl | 10.24 | 2.49 | 52 | 97.0 | 0.404 |
| | carboxylate | | | | | |
| 12 | fluorene-9-methyl carboxylate-9-ethyl | 14.81 | 4.81 | 50 | 97.0 | 0.415 |
| | carboxylate | | | | | |
| 13 | dimethyl fluorene-9,9-dicarboxylate | 13.74 | 2.91 | 53 | 98.6 | 0.378 |
| 14 | diethyl fluorene-9,9-dicarboxylate | 16.50 | 3.04 | 65 | 98.2 | 0.381 |
| 15 | di-n-propyl fluorene-9,9-dicarboxylate | 11.19 | 2.59 | 49 | 98.0 | 0.404 |
| 16 | diisopropyl fluorene-9,9-dicarboxylate | 5.35 | 4.07 | 46 | 96.9 | 0.359 |
| 17 | di-n-butyl fluorene-9,9-dicarboxylate | 8.47 | 3.10 | 48 | 98.3 | 0.384 |
| 18 | diisobutyl fluorene-9,9-dicarboxylate | 11.69 | 3.47 | 51 | 97.5 | 0.374 |
| 19 | dibenzyl fluorene-9,9-dicarboxylate | 7.79 | 3.11 | 42 | 97.4 | 0.385 |
| 20 | dimethyl fluorene-9,9-dicarboxylate | 11.19 | 2.48 | 63 | 98.0 | 0.400 |
| 21 | diethyl fluorene-9,9-dicarboxylate | 9.85 | 2.83 | 69 | 97.9 | 0.392 |
| 22 | dimethyl fluorene-9,9-dicarboxylate | 7.75 | 3.10 | 51 | 98.3 | 0.410 |
| 23 | diethyl fluorene-9,9-dicarboxylate | 10.24 | 2.91 | 60 | 98.1 | 0.408 |

The polymerization results of the above table show that, using fluorene diacid ester selected from unsaturated ring-substituted diacid ester compounds as internal electron donor and using catalysts obtained according to four different catalyst preparation processes for propylene polymerization, high activity level can be achieved, and the polypropylene prepared under the standard polymerization conditions with the aid of methyl cyclohexyl dimethoxysilane external electron donor has an isotacticity generally higher than 97%, indicating that the type of compounds can be used as the internal electron donor to in many common catalyst preparation routes, allowing the catalysts to have excellent performance of polymerization and obtain high catalytic activity and a polypropylene product having high isotacticity.

Although the above has described the present invention with the general and specific embodiments in detail, on the basis of the present invention, it is obvious for those skilled in this art to make certain modifications or improvements. Therefore, these modifications or improvements made without departing from the spirit of the present invention belong to the scope of the invention as claimed.

### INDUSTRIAL APPLICABILITY

The present invention provides a catalyst component for olefin polymerization, which comprises Mg, Ti, a halogen and an electron donor. The electron donor is selected from at least one of unsaturated ring-substituted diacid ester compounds of the general formula (I). Also provided is a catalyst containing the catalyst component and the application of the catalyst in reactions of olefin polymerization reactions, particularly in the reaction of propylene polymerization. The compound with a specific unsaturated ring-substituted structure contained in the catalyst component of the present invention has a steric hindrance effect and is capable of determining the spatial configuration of ether and acid ester functional groups, which has a positive influence in the formation of an active center of the catalyst and the improvement of the stereo specificity of the catalyst. The present invention has industrial applicability.

1. A catalyst component for olefin polymerization, comprising: Mg, Ti, a halogen and an electron donor, the electron donor being selected from at least one of unsaturated ring-substituted diacid ester compounds of the general formula (I): 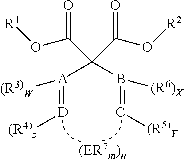
wherein, A, B, C, D, and E are carbon atoms or heteroatoms selected from N, O and S; W, X, Y, Z, and m are each 0 or 1; with the proviso that
when n is equal to 0:
I) A, B, C and D are each carbon atoms, X, Y, Z and W are each 1; or
II) A is nitrogen atom, B, C and D are each carbon atoms, W is 0, X, Y and Z are each 1; or
III) A and D are each nitrogen atoms, B and C are each carbon atoms, W and Z are each 0, X and Y are each 1; or
IV) D is a nitrogen atom, A, B, and C are each carbon atoms, Z is 0, W, X and Y are each 1; or
when n is equal to 1:
i) A, B, C, D and E are each carbon atoms, m is 2, W, X, Y, and Z are each 1; or
ii) E is a nitrogen atom, A, B, C and D are each carbon atoms, m is 1, W, X, Y and Z are each 1; or
iii) E is an oxygen atom, A, B, C and D are each carbon atoms, m is 0, W, X, Y and Z are each 1; or
iv) E is a sulfur atom, A, B, C, and D are each carbon atoms, m is 0, W, X, Y, and Z are each 1; or
v) D and E are each nitrogen atoms, A, B and C are each carbon atoms, m is 1, W, X and Y are each 1, Z is 0.
R¹ and R² are the same or different C₁-C₂₀ hydrocarbon group; R³-R⁷ are the same or different, and each are selected from a hydrogen atom, a halogen atom, an oxygen atom, a sulfur atom and a C₁-C₂₀ hydrocarbon group; and
said R¹-R⁷ optionally contain one or more R atoms as a substituent of carbon atom, a hydrogen atom, or both, where R is a heteroatom, a linear or branched C₁-C₂₀ alkyl, C₃-C₂₀ cycloalkyl, C₆-C₂₀-aryl, C₇-C₂₀ alkaryl or C₇-C₂₀ aralkyl group; wherein any two groups of R¹-R⁷ may be bonded to each other to generate one or more spiro ring or fused ring structures.

2. The catalyst component for olefin polymerization according to claim 1, wherein the compounds of the general formula (I) are of the following general formula (II): 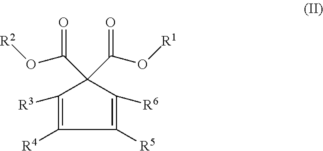
wherein R¹-R⁶ groups are defined as the general formula (I).

3. The catalyst component for olefin polymerization according to claim 2, wherein compounds of the general formula (II) are of the following general formula (III): 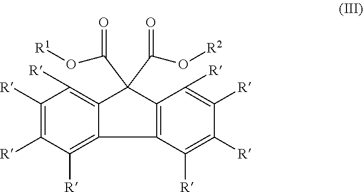
wherein R¹-R² groups are defined as in the general formula (I), R′ are same or different, and are each independently selected from the group consisting of hydrogen, a halogen, linear or branched C₁-C₂₀ alkyl, C₃-C₂₀ cycloalkyl, C₆-C₂₀ aryl, C₇-C₂₀ alkaryl, and C₇-C₂₀ aralkyl group.

4. The catalyst component for olefin polymerization according to claim 1, wherein said compounds of the general formula group (I) are selected from the group consisting of the following compounds:
diethyl 3,5-di phenyl 2H pyrrole-2,2-dicarboxylate; diethyl 3-(3-chlorophenyl)-5-methyl-pyrrole-2,2-dicarboxylate; diethyl 3-(3-bromophenyl)-5-methyl-pyrrole-2,2-dicarboxylate; diethyl-3-3-(p-chlorophenyl)-5-phenyl-2H-pyrrole-2,2-dicarboxylate; dimethylfluorene-9,9-dicarboxylate; diethylfluorene-9, 9-dicarboxylate; di-n-propyl fluorene-9,9-dicarboxylate; diisopropyl fluorene-9,9-dicarboxylate; di-n-butyl fluorene-9, 9-dicarboxylate; di-isobutyl fluorene-9,9-dicarboxylate; di-n-pentyl fluorene-9,9-dicarboxylate; di-n-hexyl fluorene-9,9-dicarboxylate; di-n-heptyl fluorene-9,9-dicarboxylate; di-n-octyl fluorene-9,9-dicarboxylate; 9-methyl carboxylate-9-ethyl carboxylate-fluorene; 9-methyl carboxylate-9-n-propyl carboxylate-fluorene; 9-methyl carboxylate-9-isopropyl carboxylate-fluorene; 9-methyl carboxylate-9-n-butyl carboxylate-fluorene; 9-methyl carboxylate-9-isobutyl carboxylate-fluorene; 9-ethyl carboxylate-9-n-propyl carboxylate-fluorene; 9-ethyl carboxylate-9-isopropyl carboxylate-fluorene; 9-ethyl carboxylate-9-n-butyl carboxylate-fluorene; 9-ethyl carboxylate-9-isobutyl carboxylate-fluorene-dimethyl 4H-benzo[g]thia<2,3-e> indazole-4,4-dicarboxylate; diethyl-5-phenyl-3 (p-toluene)-2H-pyrrole-2,2-carboxylate; diethyl-3 (p-methoxyphenyl)-5-phenyl-2H-pyrrole-2,2-dicarboxylate; diethyl 5-(p-nitro)-3-phenyl-2H-pyrrole-2,2-dicarboxylate; diethyl-2,3-diphenyl-1H-indene-1,1-dicarboxylate; diethyl-2-phenyl-1H-indene-1,1-dicarboxylate; di ethyl-2-(4-chlorophenyl)-1H-indene-1,1-dicarboxylate; diethyl-2-(4-methoxyphenyl)-1H-indene-1,1-dicarboxylate; dimethyl 3-(4-methylphenyl)-2-phenyl-1H-indene-1,1-dicarboxylate; dimethyl-3-(4-nitrophenyl)-1 H-indene-1,1-dicarboxylate; dimethyl amino-4-pentamethoxycarbonyl-1,2,3,5,5-pentamethoxycarbonylcyclopentadiene; methyl 3-phenyl-indene-1,1-dicarboxylate; dimethyl-5-(p-chlorophenyl)3-phenyl-2H-pyrrole-2,2-dicarboxylate; dimethyl 3,4-di(p-chlorophenyl) 2H-pyrrole-dicarboxylate; dimethyl 3-(p-nitrophenyl)-5-phenyl-2H-pyrrole-2,2-dicarboxylate; dimethyl 3-(m-nitrophenyl)-5-phenyl-2H-pyrrole-2,2-dicarboxylate; dimethyl 5-(m-nitrophenyl)-5-phenyl-2H-pyrrole-2,2-dicarboxylate; dimethyl 5,6-dimethyl-5H,6H-cyclopentadiindole-11,11-dicarboxylate; 1-(2-nitrophenylthio)-2,3,4,5,5-methyl carboxylate-cyclopentadiene; 1-(2,4-dinitrophenyl)-2,3,4,5,5-methyl pentacarboxylate-cyclopentadiene; methyl-2-tert-butyl-3-methyl-1H-indene-1,1-dicarboxylate; dimethyl 3-methyl-2-trimethylsilyl-indene-1,1-dicarboxylate; dimethyl 3-methyl-2-phenyl-indene-1,1-dicarboxylate; diethyl-2,3-di-n-propyl-1H-indene-1,1-dicarboxylate; dimethyl-3-hydroxymethyl-2-phenyl-1H-indene-1,1-dicarboxylate; dimethyl-2-tert-butyl-5, 6-dimethoxy-3-methyl-1H-indene-1,1-dicarboxylate; dimethyl-2-phenyl-3-(thia-2-yl)-1H-indene-1,1-dicarboxylate; dimethyl-3-(2-toluene)2-phenyl-1H-indene-1,1-dicarboxylate; dimethyl 3-(2-methoxycarbonylphenyl)-2-phenyl-1H-indene-1,1-dicarboxylate; dimethyl 3-(4-trifluoromethylphenyl) 2-phenyl-1H-indene-1,1-dicarboxylate; dimethyl 3-(4-acetylphenyl) 2-phenyl-1H-indene-1,1-dicarboxylate; dimethyl-2-(cyclohex-1-enyl)-3-(4-acetylphenyl)-1H-indene-1,1-dicarboxylate; dimethyl 2-[(ethoxycarbonyl)methyl]-1H-indene-1,1-dicarboxylic acid ester; 1,1-diethyl-1H-indene-1,1-dicarbonate; ethyl 7-chloro-5methyl-pyrazolo[4,3-d]pyrimidine-3,3-dicarbonate; ethyl 5-chloro-7-methyl-pyrazolo[4, 3-d]pyrimidine-3,3-dicarbonate; ethyl 5-amino-7-methyl-pyrazolo[4,3-d]pyrimidine-3,3-dicarbonate; ethyl 7-methoxy-5-methyl-pyrazolo[4,3-d]pyrimidine-3,3-di carbonate; 1-p-methyl phenyl amino-2,3,4,5,5-pentamethoxycarbonylcyclopentadiene; dimethyl-3H-phenanthro<9,10-c>pyrazole-3,3-dicarbonate; 3,3-di(methoxycarbonyl)-3H-indazole; 3,3-di(ethoxycarbonyl)3H-indazole; 1-trichloromethyl-2,3,4,5,5-pentamethoxycarbonylcyclopentadiene; 1-(2-methyl-4-nitrophenyl)-pentamethoxycarbonylcyclopentadiene; 1-(2-iodo-4-nitrophenyl)-pentamethoxycarbonylcyclopentadiene; 2-(2-iodo-4-nitrophenyl)-1,3,4,5,5-pentamethoxycarbonylcyclopentadiene; 1-(2,4-dinitrophenyl)-2,3,4,5,5-pentamethoxycarbonylcyclopentadiene; 4-benzyl-1,2,3,5,5-penta(methoxycarbonyl)cyclopentadiene; 3-benzyl-1,2,4,5,5-penta(methoxycarbonyl)cyclopentadiene; 2-(trifluoromethyl)-5-carbonyl-3,3-di(methoxycarbonyl)-3H-indole; 2-(trifluoromethyl)-5-carbonyl-7-methyl-3,3-di(methoxycarbonyl)-3H-indole; 3-(trifluoromethyl)-5-hydroxy-7-methoxy-3,3-di(methoxycarbonyl)-3H-indole; diethyl-3-phenyl-5 (p-toluene)2H-pyrrole-2,2-dicarbonate; diethyl-2-(4-chlorophenyl)-5-morpholine-4H-imidazole-4,4-dicarbonate; 4,5,5-methyl tricarboxylate-1,2,3-trichlorocyclopentadiene; methyl-3-methyl-4-trimethylsilyl-cyclopenta-2,4-diene-1,1-dicarbonate; diethyl-2, 5-diphenyl-4H-imidazole-4,4-dicarbonate; diethyl-3-benzyl-2-phenyl-1H-indene-1,1-dicarbonate; diethyl-3-(4-(methoxycarbonyl)phenyl)2-phenyl-1H-indene-1,1-dicarbonate; diethyl-3-(4-acetylphenyl)2-phenyl-1H-indene-1,1-dicarbonate; diethyl-2-methoxymethyl-1H-indene-1,1-dicarbonate; dimethyl-2-tert-butyl-1H-indene-1,1-dicarbonate; diethyl-2-tert-butyl-1H-indene-1,1-dicarbonate; dimethyl 2-n-butyl-1H-indene-1,1-dicarbonate; diethyl 2-n-butyl-1H-indene-1,1-dicarbonate; diethyl 2-n-hexyl-1H-indene-1,1-dicarbonate; diethyl-2-(3-cyano-1-propyl)-1H-indene-1,1-dicarbonate; diethyl-2-diethoxymethyl-1H-indene-1,1-dicarbonate; diethyl-2-(4-methoxyphenyl)-1H-indene-1,1-dicarbonate; diethyl-2-(1-cyclohexene)-1H-indene-1,1-dicarbonate; diethyl-2-(cyclohexyl)-1H-indene-1,1-dicarbonate; diethyl-3-(3-toluene)-2-phenyl-1H-indene-1,1-dicarbonate; diethyl-3-(3-nitrophenyl)-2-phenyl-1H-indene-1,1-dicarbonate; diethyl 13H-indeno[1,2-e]-phenanthrene-13,13-dicarbonate; diethyl-2-hexyl-3-(4-methoxyphenyl)1H-indene-1,1-dicarbonate; ethyl cyclopenta[c]thia-5,5-dicarbonate; 4-[4-[4-(methylsulfonic)phenyl]1,1-di(methoxy)cyclopenta-2,4-diene-3-yl]pyridine; fluorene-4,9, 9-dicarboxylic acid-4-tert-butyl-9,9-dimethyl ester; methyl 4-(4-amino-pyridine-3-ylcarbamoyl)-fluorene-9,9-dicarbonate; dimethyl 4-(3-amino-pyridine-4-ylcarbamoyl)-fluorene-9,9-dicarbonate; diethyl-3-iodo-2-phenyl-1H-indene-1,1-dicarbonate; diethyl-3-iodo-2-n-pentyl-1H-indene-1,1-dicarbonate; diethyl-3-iodo-2-(3methoxyphenyl)-1H-indene-1,1-dicarbonate; diethyl-3-iodo-2-(naphthalen-2-yl)-1H-indene-1,1-dicarbonate; di-n-hexyl-fluorene-9,9-dicarbonate; di-n-heptyl-fluorene-9, 9-dicarbonate; diethyl-2-phenyl-3H-indene-3,3-dicarbonate; di ethyl-2-bromo-1H-indene-1,1-dicarbonate;
1-ethyl-1-methyl-cyclohexa-2, 5-diene-1,1-dicarbonate; N,4,4-triethoxycarbonyl-1,4-dihydro-pyridine; 2,6-diphenyl-4,4-dimethoxycarbonyl-4H-pyrane; 2,6-diphenyl-4,4-dimethoxycarbonyl-1,4-dihydropyridine; 2,6-di(4-chlorophenyl)-4,4-dimethoxycarbonyl-4H-pyrane; 2,6-di(4-methoxyphenyl)-4,4-dimethoxycarbonyl-4H-pyrane; 2,6-di(4-chlorophenyl)-4,4-dimethoxycarbonyl-1,4-dihydropyridine; 2,6-di(4-methoxyphenyl)-4,4-dimethoxycarbonyl-1,4-dihydropyridine; 1-cyclopentyl-4,4-di(methoxycarbonyl)-1,4-dihydropyridine; 1-n-hexyl-4,4-di(methoxycarbonyl)-1,4-dihydropyridine; 1-methoxy-6, 6-dimethyl carbonyl oxymethyl-cyclohexa-1,4-di ene; dimethyl 1,4-dihydronaphthalene-1,1-di carbonate; 2,6-di(4-chlorophenyl)-4,4-dimethoxycarbonyl-4H-thiapyran; diethyl-3-bromo-1,4-dihydro-1-methylpyridazino[3,4-b]quinoxaline-4,4-dicarbonate; diethyl-5-bromo-3-phenyl-1,4-dihydropyridazine-4,4-dicarbonate; trihexyl-3-phenyl-1,4-dihydropyridazine-4,4,5-tricarboxylate; 1-phenethyl-di(m ethoxycarbonyl)1,4-dihydropyridine; diethyl-2-methyl-6-benzo(4H-pyran)4,4-dicarbonate; 1-(2-naphthylmethyl)-4,4-di(m ethoxycarbonyl)-1,4-dihydropyridine; dimethyl-3-acetyl-1-methylquinoline-4,4(1H)-dicarbonate.

5. The catalyst component for olefin polymerization according to claim 1, comprising the reaction product of a titanium compound, a magnesium compound and an unsaturated ring-substituted diacid ester compound selected from the general formula (I), the precursor of said magnesium compound is selected from at least one of: Mg(OR)₂, XₙMg(OR)₂₋ₙ, MgCl₂.mROH, R₂₋ₙMgXₙ, MgR₂, MgCl₂/SiO₂, MgCl₂/Al₂O₃, or mixture of magnesium halide and titanium alkoxide, wherein m is a number from 0.1 to 6, 0<n<2, X is halogen, R is hydrogen or C₁-C₂₀ hydrocarbon group; said titanium compound is represented by the general formula TiXn(OR)₄₋ₙ, wherein R is C₁-C₂₀ hydrocarbon group, X is halogen, n=1-4.

6. A catalyst for olefin CH₂═CHR polymerization, wherein R is hydrogen or a hydrocarbon group having 1-12 carbon atoms, the catalyst comprising the reaction product of the following substances:
(a) a catalyst component according to claim 1;
(b) at least one organic aluminum compound of the general formula AlRₙX₍₃₋ₙ₎, wherein R is hydrogen, hydrocarbon group having 1-20 carbon atoms; X is halogen, n is an integer of 0≦n≦3; and optionally,
(c) at least one external electron donor compound.

7. The catalyst according to claim 6, wherein the organic aluminum compound (b) is a trialkylaluminum compound.

8. The catalyst according to claim 7, wherein the trialkylaluminum compound is selected from the group consisting of trimethylaluminum, triethylaluminum, triisobutylaluminum, tri-n-butyl aluminum, tri-n-hexyl aluminum, and trioctyl aluminum.

9. The catalyst according to claim 6, wherein said external electron donor compound (c) is selected from a siloxane compound of the general formula RₙSi(OR₁)₄₋ₙ, wherein R and R₁ are each a C₁-C₁₈ hydrocarbon group, optionally substituted by heteroatoms; n is an integer of 0≦n≦3.

10. A pre-polymerization catalyst for olefin CH₂═CHR polymerization, wherein R is hydrogen or a hydrocarbon group having 1 to 12 carbon atoms, wherein said pre-polymerization catalyst comprises a prepolymer obtained by pre-polymerization of the catalyst component according to claim 1 and an olefin.

11. The pre-polymerization catalyst according to claim 10, wherein the olefin for pre-polymerization is ethylene or propylene.
